(19) United States
(12) Patent Application Publication
(10) Pub. No.: US 2011/0005915 A1
(43) Pub. Date: Jan. 13, 2011

(54) METHOD FOR RECOVERING FLUOROCARBOXYLIC ACIDS

(75) Inventors: Claude Mercier, Jonquieres (FR); Francois Metz, Irigny (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Rhodia Operations, Aubervilliers Cedex (FR)

(21) Appl. No.: 12/680,496

(22) PCT Filed: Sep. 25, 2008
(86) PCT No.: PCT/EP08/62852

§ 371 Date: Sep. 24, 2010

(30) Foreign Application Priority Data
Sep. 28, 2007 (FR) 07/06829

(51) Int. Cl. C07C 51/46; C07C 51/44; B01D 3/36; C07C 53/18

(52) U.S. Cl. 203/51; 203/50; 203/61

(57) ABSTRACT

The invention relates to a method for recovering fluorocarboxylic acids from aqueous compositions containing said acids. The invention more particularly relates to the recovery of flourocarboxylic acids forming an azeotrope with water by contact with a strong acid.

The present invention relates to a method of recovery of fluorocarboxylic acids from aqueous solutions containing said acids. The invention relates more particularly to the recovery of fluorocarboxylic acids forming an azeotrope with water, by contact with a strong acid.

The chemical industry uses fluorocarboxylic acids in many fields of activity for the most part they are soluble in water and are often present in varying amounts in industrial effluents, notably wastewater.

In view of the costs of these fluorocarboxylic acids, it can prove useful and profitable to treat the aqueous effluents containing said acids, in order to recover them.

Moreover, the presence of fluorocarboxylic acids in industrial aqueous effluents may be harmful to the environment and as environmental requirements are becoming increasingly stringent, the recovery of fluorocarboxylic acids is now becoming a problem for which no acceptable solution is yet available on an industrial scale.

A simple and effective means might comprise distillation of the aqueous waste containing the fluorocarboxylic acid or acids. However, these acids generally form azeotropes with water, so that their distillation to a satisfactory degree of purity is impossible. For example, trifluoroacetic acid (TEA) forms an azeotrope with water, with boiling point of about 103° C. This temperature, which is very close to the boiling point of water, makes simple TFA/water distillation even less effective, and it cannot be used.

Thus, one aim of the invention is to propose a simple and effective method permitting the recovery of fluorocarboxylic acids from aqueous effluents, with an efficiency and a degree of purity that are satisfactory and economically profitable, notably on an industrial scale.

Other aims will become clear from the following account of the present invention.

As already mentioned, a conventional method of distillation does not allow the recovery of a fluorocarboxylic acid of high purity, because of the azeotrope that said forms with water. The inventors have now discovered a method, which is the object of the invention, which permits the recovery of fluorocarboxylic acids present is aqueous effluents (or more simply solutions).

Thus, the present invention relates firstly to a method of recovery of fluorocarboxylic acid(s) from an aqueous effluent, characterized in that it comprises the following stages:
a) contacting said aqueous effluent comprising at least one fluorocarboxylic acid with at least one strong acid;
b) distillation of the mixture obtained in stage a);
c) recovery of the distillate from stage b) consisting essentially of said fluorocarboxylic acid.

It has in fact been discovered that strong acid mixed with the aqueous effluent makes it possible to “break” the fluorocarboxylic azeotrope, making the distillation of said fluorocarboxylic acid possible, effectively and profitably from the standpoint of yield and purity.

Aqueous effluent means any residue, solution, discharge, industrial or other, comprising from 5% to 70 wt. %, preferably from 10 to 50 wt. % of at least one fluorocarboxylic acid relative to the total weight of the effluent to be treated.

The remainder or the effluent is constituted of water but can in addition comprise from 0 to 20 wt. %, preferably from 0 to 15 wt. % of impurities, relative to the weight of fluorocarboxylic acid(s). These impurities can be of any kind, and are generally constituted of residual impurities from processes for preparation of chemicals, such as notably organic solvents, excess reactants, synthesis intermediates, reaction byproducts, etc.

Thus, the amount of water present in the effluents to be treated is generally between 15 and 95 wt. % preferably from 40 to 80 wt. %, relative to the total weight of the effluent.

The fluorocarboxylic acids that can be recovered according to the method of the invention are more particularly the aliphatic fluorocarboxylic acids, which form an azeotrope with water.

These fluorinated carboxylic acids preferably possess a linear or branched aliphatic chain, having from 1 to 10 carbon atoms in total and corresponding to the following general formula (AF):

CₙFₘHₚ−COOH (AF)

in which:
n is integer, such that 1≦n≦9;
m is an integer, such that 1≦m≦2n+1; and
p is an integer, such that 0≦p≦2n, with p+m=2n+1.

According to a preferred embodiment of the invention, the fluorocarboxylic acids are perfluorocarboxylic acids, i.e. acids of formula (AF) for which m=2n+1 and p=0. According to another embodiment, the fluorocarboxylic acids are fluorinated carboxylic acids in which all of the fluorine atoms are carried exclusively by the carbon atom in position relative to the carboxyl group (COOH), in particular the acids of formula F₃C−(—CH₂—)ₙ₋₁—COOH, where n is as defined in the above formula (AF).

It should be noted that the fluorocarboxylic acids can also comprise halogen atoms other than fluorine, such as chlorine or bromine.

Examples of fluorocarboxylic acids that can be recovered by the method of the invention are difluoroacetic acid (or DFA, F₂HC—COOH), chlorodifluoroacetic acid (or CDFA, ClF₂C—COOH), trifluoroacetic acid (or TFA, F₃C—COOH),3,3,3-trifluoropriopionic acid (F₃C—CH₂—COOH), pentafluoropropionic acid (F₅C₂—COOH), heptafluorobutyric acid (F₇C₃—COOH), perfluoropentanoic acid (F₉C₄—COOH), perfluorohexanoic acid (F₁₁C₅—COOH), perfluoroheptanoic acid (F₁₃ —COOH), and perfluorooctanoic acid (F₁₅C₇—COOH). Trifluoroacetic acid (TFA), which forms a positive azeotrope (boiling point: 103° C.) naturally with water, is quite particularly preferred in the method of the present invention.

It must be understood that the aqueous effluent employed in the method can comprise one or more fluorocarboxylic acids, preferably just one, two or three, more preferably the effluent only comprises just one fluorocarboxylic acid, and quite preferably the effluent comprises only TFA.

Furthermore, the invention does not exclude the case when the fluorocarboxylic acid comes from hydrolysis of the corresponding acid anhydride.

In the method of the invention, the strong acid can be added to the aqueous effluent, or the aqueous effluent can be added to the strong acid.

The strong acid, which is thus mixed with the aqueous effluent to be treated, has the effect of “trapping” the water of said aqueous effluent, and “breaking” the water/fluorocarboxylic acid azeotrope.

The strong acids are on preferably strong protic acids, i.e. are able to liberate at least one hydrogen atom. The strong protic acids of interest for the method of the invention generally possess a pKa in water of less than 0.1, preferably less than −1, measured at 20° C. They must be inert with respect to the fluorocarboxylic acid to be recovered.

Examples of strong acids that can be used in the method of the present invention comprise sulfuric acid, oleums (at various concentrations, for example 20%, 30%, 40%), hydrochloric acid in the liquid or gaseous state (hydrogen chloride), phosphoric acid, chlorosulfuric acid, fluorosulfuric acid, perchloric acid, and sulfonic acids, for example methanesulfonic acid, trifluoromethanesulfonic acid, toluenesulfonic acid, or phenolsulfonic acid.

Nitric acid is not, however, preferred, owing to its very strong oxidizing power and its possibility of reacting with the fluorocarboxylic acid to be recovered.

It is of course also possible to use solid strong acids, notably supported acids, for example the sulfonic resins, and more particularly the resins marketed under various trade names, among which we may mention the resins Temex ® 50, Amberlyst® 15, Amberlyst® 35, Amberlyst® 36, Dowex® 50W, among others.

The aformentioned resins are constituted of a polystyrene backbone, which bears sulfonic groups as functional groups. The polystyrene backbone is obtained by polymerization of styrene and divinylbenzene, under the influence of an activation catalyst, most often an organic peroxide, resulting in a crosslinked polystyrene which is then treated with concentrated sulfuric or sulfochloric acid, resulting in a sulfonated styrene-divinylbenzene copolymer.

It is also possible to use sulfonic resins, which are phenol-formol copolymers and bear a methylenesulfonic group on the aromatic nucleus, for example the resin marketed under the name Duolite® ARC 9359.

Other resins available on the market are also suitable and we may mention the perfluorinated resins bearing sulfonic groups and more particularly Nafion®, which is a copolymer of tetrafluoroethylene perfluoro-[2-(fluorosulfonylethoxy)propyl]vinylether.

Mixtures of two or more of the aforementioned acids, whether liquid, gaseous or solid (supported), can be used, in all proportions. As a general rule, sulfuric acid, notably sulfuric acid at 98 wt. %, has shown good results, notably in the recovery of TFA.

The amount of acid(s) mixed with the effluent to be treated must be sufficient to “break” the water/fluorocarboxylic acid azeotrope. Although an excess of acid is not harmful, it is preferably avoided, for obvious reasons of cost of the method of recovery.

Thus, as a general rule, the weight ratio of acid(s) to water contained in the effluent is between 1 and 10, said ratio preferably being between 1.5 and 3.

The strong acid can be added to the aqueous effluent to be treated in one go, or in portions, for example dropwise, controlling exothermic effect resulting from this addition of strong acid to the aqueous medium.

Moreover, the aqueous effluent can be added to the strong acid an one go or in portions, with the usual precautions required when adding water to a strong acid.

Although conceivable, it is not necessary to cool the medium while mixing strong acid and aqueous effluent. In fact, when controlled, the exothermic effect can be used advantageously for heating all of the reaction mixture, or even to bring it to the boil, with a view to the next stage distillation.

The strong acid and the effluent to be treated can be mixed directly in the reactor where distillation is carried out. According to another aspect, it can be envisaged to conduct one or more campaigns of mixing of strong acid/effluent to be treated, and then charge the reactor directly with the mixture or mixtures thus prepared.

The stage of distillation of the mixture of strong acid/effluent to be treated is carried out conventionally, employing equipment and conditions known by a person skilled in the art.

It can be envisaged to carry out a first distillation, then add a further amount of strong acid and then complete the distillation.

It can also be envisaged to carry out the distillation stage as a batch process or as a continuous process. For a continuous process, it can be envisaged to add the strong acid continuously to the effluent to be treated, which forms the distillation bottom product, or add the effluent to be treated continuously to the strong acid, which forms the distillation bottom product, or alternatively add continuously, to the reactor, the previously prepared mixture of strong acid/effluent to be treated.

The distillation device is of any type that is known per se and comprises for example a reactor (a still) containing the effluent to be treated and the strong acid, said reactor being equipped with heating means, for example oil bath, sand bath, double jacket and other known means by which the mixture contained in the still can be brought to the boil (reflux conditions).

The still is also equipped with means permitting the distillation and condensation of the vapors coming from the reaction mixture under reflux. These means can take all known forms, such as distillation column, plate column, packed column etc., condenser, evaporator, of a conventional type, for example with circulation of coolant, or thin-film evaporator or wiped-film evaporator, as marketed for example by the company Luwa.

Moreover, the distillation device comprises, optionally but advantageously, means permitting stirring of the mixture to be distilled. The distillation device also comprises means for temperature measurement, advantageously for measuring the temperature of the mixture to be distilled and the temperature of the distillate. These means for temperature measurement are known by a person skilled in the art and can have visual reading, recording, or can even be controlled, notably by computer software.

As mentioned above, distillation of the mixture of aqueous effluent/strong acid is carried out conventionally, with reflux of the mixture. The distillation is generally carried out at atmospheric pressure, but it can be envisaged to work under partial vacuum or high vacuum (for example between 750 mmHg and 50 mmHg, i.e. between about 1000 hPA and 67 hPa), or alternatively under pressure. Working under inert atmosphere, for example nitrogen or argon, can also be envisaged.

For reasons of economics and convenience, however, it is preferable to carry out the distillation stage at atmospheric pressure, without using inert gas.

The specific distillation of fluorocarboxylic acid is made possible by the presence of the strong acid, which makes it possible to “break” the azeotrope that forms naturally between the fluorocarboxylic acid and the water contained in the aqueous effluent.

Thus, during distillation, the fluorocarboxylic acid (or acids) distils/distil at its (their) characteristic boiling point, thus permitting recovery of said fluorocarboxylic acid (or acids) at the top of the distillation device (generally at the head of column) with a high degree of purity. In the preferred case of trifluoroacetic acid (TFA), it is thus possible to distil this acid at its characteristic boiling point, i.e. about 72° C., at atmospheric pressure. In the case of the fluorocarboxylic acids whose characteristic boiling point is higher than that of water (100° C. at atmospheric pressure), the water is distilled first, then the fluorocarboxylic acid whose characteristic boiling point is higher than that of water.

“High degree of purity” means purity greater than or equal to 95 wt. %, preferably greater than or equal to 99 wt. %.

The method of the invention also offers the advantage that it is easy to implement and requires little capital expenditure, while offering a yield in recovery of fluorocarboxylic acid that is acceptable, and even satisfactory.

Thus, the yield in recovery of fluorocarboxylic acids is generally above 50 wt. %, preferably above 75 wt. %, and can even reach values above 90 wt. %, or even above 95 wt. %.

At the end of the distillation stage, the reactor (or still) contains a reside comprising the strong acid that was used for breaking the azeotrope, and optionally water, if the latter was not distilled before or after distillation of the fluorocarboxylic acid. Of course, the residual strong acid can advantageously be recycled and used again in a new process of recovery of fluorocarboxylic acid.

According to another aspect, the invention relates to a distillation device, said device comprising:
a reactor (a still) containing a reaction mixture comprising an aqueous effluent comprising from 5 to 70 wt. %, preferably from 10 to 50 wt. % of at least one fluorocarboxylic acid relative to the total weight of said effluent, and at least one strong acid, the weight ratio of acid(s) to water contained in the effluent being between 1 and 10;
heating means;
means for distillation and condensation of the vapors coming from the reaction mixture under reflux;
means for temperature measurement and/or control, and optionally means for stirring the reaction mixture, or means for controlling the boiling of said mixture.

The present invention also relates to an industrial installation comprising the distillation device previously defined, means for storage and/or for receiving aqueous effluents containing at least one fluorocarboxylic acid and at least one strong acid, as defined previously, as well as means for recovery (and optionally storage) of fluorocarboxylic acid(s) obtained from the distillation device, and means for discharge (and optionally storage) of the aqueous effluents and acids that form the waste products from the distillation device.

The present invention is now illustrated by means of the following examples, which are not in any way intended to limit the scope of protection of the present invention, which is defined by the accompanying claims.

### EXAMPLES

The procedure used in the examples will now be described.

Use a reactor (glass or stainless steel, for example) equipped with a distillation column, a cooling apparatus (0° C.), a pouring funnel and a moisture trap (CaCl₂). The reactor is placed in an oil bath equipped with a thermometer and a mechanical stirrer.

Charge the reactor with the aqueous industrial effluent containing a fluorocarboxylic acid.

Add sulfuric acid at 98 wt. % dropwise to the fluorocarboxylic acid, without heating.

After adding all of the sulfuric acid, heat the reactor by means of the oil bath.

Bring the reaction mixture to reflux. The temperature at the head of the column rises from room temperature to the temperature of the boiling point of the fluorocarboxylic acid to be recovered.

Maintain total reflux for 30 min, then begin to withdraw the distillate at the temperature of the boiling point of the fluorocarboxylic acid at the top of the column with a reflux ratio between 1 and 5, preferably at least 3 to 4.

When almost all of the fluorocarboxylic acid has been distilled, increase the temperature inside the reactor, and maintain this temperature until the temperature of the vapors decreases. Then stop the withdrawal of fluorocarboxylic acid.

Cool the reactor to room temperature and pour the residue into a cask for recovery of effluents.

### Example 1

Load 250 g of an aqueous industrial effluent containing 30 wt. % of trifluoroacetic acid (TFA), 2.4 wt. % of toluene, and 1 wt. % of pyridine, in a 500 ml reactor equipped with a magnetic stirrer, heating means, a distillation device and a thermometer.

Heat the reactor to total reflux for at least 30 min (bottom temperature: 102° C.; head temperature: 100° C., then add 300 g of sulfuric acid dropwise.

The temperature of the vapors decreases to 72° C. and withdrawal of the distillate at 72° C. from the head of column is started.

When the head temperature increases (disappearance of reflux), recovery of the distillate is stopped.

In this way, 55 g of trifluoroacetic acid is recovered (yield 73%), with a degree of purity greater than 99.8%, and a moisture level of 357 ppm measured by the Karl Fischer method.

### Example 2

A second test is carried out, following the procedure described in example 1, mixing 215 g of an aqueous solution at 29 wt. % trifluoroacetic acid with 322 g of sulfuric acid at 98 wt. %.

We recover 45.15 g of trifluoroacetic acid (yield 73%) with a degree of purity greater than 99.8%, and a moisture level of 403 ppm measured by the Karl Fischer method.

### Example 3

A third test is carried out, following the procedure described in example 1, mixing 200 g of an aqueous solution at 30 wt. % of trifluoroacetic acid, containing 10.65 g of toluene, with 241 g of sulfuric acid at 98 wt. %.

We recover 47.7 g of trifluoroacetic acid (yield 79.2%) with a degree of purity greater than 99.8%, and a moisture level of 260 ppm measured by the Karl Fischer method.

### Example 4

An aqueous effluent is used with 30 wt. % of TFA, and sulfuric acid at 98% (Sinopharm, grade A.R).

Load 244.2 g of sulfuric acid in a 500 ml reactor, equipped with a distillation column, than add cautiously, dropwise, 204.7 g aqueous effluent containing 30 wt. % of trifluoroacetic acid, while beginning to heat the assembly.

Reflux appears at 72° C. at the head of column, whereas the reactor temperature drops to 126° C.

The temperature at the head of column stabilizes at 72° C. and recovery of pure TFA begins, and the bottom temperature increases slowly to 138° C., this temperature being maintained until distillation of TFA stops.

We recover 45.1 g of TFA (yield 73.7%) with a degree of purity greater than 99.8%.

### Examples 5 to 14

Corresponding tests are carried out following the procedure described in example 1, mixing 215 g of an aqueous solution a x wt. % of perhalogenated carboxylic acid with y g of mineral acid at z wt. %.

The results obtained are presented in Table (I).

TABLE I

| | | | | Weight | | | |
|---|---|---|---|---|---|---|---|
| | | | | of | Degree | | |
| | | | | mineral | of | | Moisture |
| | Perhalogenated | Concentration, | Mineral | acid g | recovery | Purity | level |
| Ex | acid | wt. % (x) | acid (z) | (y) | (%) | % | % |
| 5 | CF₃CO₂H | 10 | H₂SO₄ | 355 | 39.1 | 98 | 1 |
| | | | (98%) | | | | |
| 6 | | 28 | H₂SO₄ | 158 | 38.3 | 99 | — |
| | | | (98%) | | | | |
| 7 | | 28 | H₂SO₄ | 474 | 93.6 | 99.6 | 0.05 |
| | | | (98%) | | | | |
| 8 | | 50 | H₂SO₄ | 197 | 91.3 | 99.8 | 0.1 |
| | | | (98%) | | | | |
| 9 | | 28 | PPA* | 135 | 45.4 | 99.4 | 0.1 |
| | | | (80%) | | | | |
| 10 | | 28 | P₂O₅ | 309 | 49.1 | 98.9 | 0.67 |
| | | | (100%) | | | | |
| 11 | CClF₂CO₂H | 30 | H₂SO₄ | 461 | 25.8 | 92.5 | 7.5 |
| | | | (98%) | | | | |
| 12 | CF₃CF₂CO₂H | 30 | H₂SO₄ | 276 | 54.4 | 98.8 | 38 |
| | | | (98%) | | | | ppm |
| 13 | CF₃ (CF₂) ₂CO₂H | 30 | H₂SO₄ | 353 | 57 | 95 | 5 |
| | | | (98%) | | | | |
| 14 | CF₃ (CF₂) ₂CO₂H | 30 | H₂SO₄ | 691 | 43 | 96.3 | 0.38 |
| | | | (98%) | | | | |

* PPA = polyphosphoric acid (80% P₂O₅)

1-19. (canceled)

20. A method for recovery of fluorocarboxylic acid(s) from an aqueous effluent, comprising the following steps:
a) contacting said aqueous effluent comprising at least one fluorocarboxylic acid with at least one strong acid:
b) distilling the mixture obtained in step a);
c) recovering a distillate from step b) consisting essentially of said fluorocarboxylic acid.

21. The method of claim 20, wherein step a) comprises adding said strong acid to said aqueous effluent.

22. The method of claim 20, wherein step a) comprises adding said aqueous effluent to said strong acid.

23. The method of claim 20, wherein the aqueous effluent comprises from 5 to 70 wt. % of at least one fluorocarboxylic acid relative to the total weight of said effluent.

24. The method of claim 23, wherein the aqueous effluent comprises from 10 to 50 wt. % of at least one fluorocarboxylic acid relative to the total weight of said effluent.

25. The method of claim 20, wherein the aqueous effluent comprises from 0 to 20 wt. % of impurities, relative to the weight of fluorocarboxylic acid(s).

26. The method of claim 25, wherein the aqueous effluent comprises from 0 to 15 wt. % of impurities, relative to the weight of fluorocarboxylic acid(s).

27. The method of claim 20, wherein at least one fluorocarboxylic acid comprises aliphatic fluorocarboxylic acids forming an azeotrope with water and comprising from 1 to 10 total carbon atoms.

28. The method of claim 27, wherein said aliphatic fluorocarboxylic acids comprise a linear or branched aliphatic chain.

29. The method of claim 20, wherein at least one fluorocarboxylic acid comprises a perfluorocarboxylic acid or a fluorocarboxylic acid wherein all of the fluorine atoms are carried exclusively by the carbon atom in position ω relative to the carboxyl group.

30. The method of claim 20, wherein at least one fluorocarboxylic acid comprises difluoroacetic acid, chlorodifluoroacetic acid, trifluoroacetic acid, 3,3,3-trifluoropriopionic acid, pentafluoropropionic acid, heptafluorobutyric acid, perfluoropentanoic acid, perfluorohexanoic acid, perfluoroheptanoic acid, or perfluorooctanoic acid.

31. The method of claim 20, wherein at least one fluorocarboxylic acid comprises trifluoroacetic acid.

32. The method of claim 20, wherein the pKa in water of said strong acid is below 0.1 at 20° C.

33. The method of claim 32, wherein the pKa in water of said strong acid is below −1 at 20° C.

34. The method of claim 20, wherein the strong acid comprises sulfuric acid, oleums, hydrochloric acid in the liquid or gaseous state, phosphoric acid, chlorosulfuric acid, fluorosulfuric acid, perchloric acid, sulfonic acids, supported acids, including sulfonic resins, sulfonated styrene-divinylbenzene copolymers, phenol-formol copolymeric sulfonic resins bearing a methylenesulfonic group on the aromatic nucleus, perfluorinated resins bearing sulfonic groups, copolymers of tetrafluoroethylene and perfluoro-[2-(fluorosulfonylethoxy)propyl]vinylether, or mixtures thereof.

35. The method of claim 34, wherein the concentration of said oleums is 20%.

36. The method of claim 34, wherein the concentration of said oleums is 30%.

37. The method of claim 34, wherein the concentration of said oleums is 40%.

38. The method a claim 34, wherein said sulfonic acids comprise methanesulfonic, trifluoromethanesulfonic, toluenesulfonic, phenolsulfonic acids, or mixtures thereof.

39. The method of claim 20, wherein the strong acid comprises sulfuric acid.

40. The method of claim 39, wherein the strong acid comprises 98 wt. % sulfuric acid.

41. The method of claim 20, wherein the weight ratio of acid(s) to water in the effluent ranges from 1 to 10.

42. The method of claim 41, wherein the weight ratio of acid(s) to water in the effluent ranges from 1.5 to 3.

43. The method of claim 20, comprising carrying out distillation step b) at atmospheric pressure.

44. The method of claim 20, comprising recovering the fluorocarboxylic acid with a degree of purity greater than or equal to 95 wt. %.

45. The method of claim 44, comprising recovering the fluorocarboxylic acid with a degree of purity greater than or equal to 99 wt. %.

46. The method of claim 20, wherein the recovery yield is greater than 50 wt. %.

47. The method of claim 46, wherein the recovery yield is greater than 75 wt. %.

48. The method of claim 47, wherein the recovery yield is greater than 90 wt. %.

49. The method of claim 48, wherein the recovery yield is greater than 95 wt. %.

50. The method of claim 20, comprising carrying out the distillation step b) as a batch operation.

51. The method of claim 20, comprising carrying out the distillation step b) continuously.

52. The method of claim 20, wherein said method is carried out with a distillation device comprising:
a reactor comprising a reaction mixture comprising an aqueous effluent comprising from 5 to 70 wt. % of at least one fluorocarboxylic acid relative to the total weight of said effluent, and at least one strong acid,
wherein the weight ratio acid(s) to water in the effluent ranges from 1 to 10;
heating means;
means for distillation and condensation of the vapors coming from the reaction mixture under reflux; and
means for temperature measurement and/or control.

53. The method of claim 52 wherein said aqueous effluent comprises from 10 to 50 wt. % of at least one fluorocarboxylic acid relative to the total weight of said effluent.
